(19) United States
(12) Patent Application Publication
(10) Pub. No.: US 2026/0199027 A1
(43) Pub. Date: Jul. 16, 2026

(54) PEDICLE SCREW ORIENTATION ESTIMATION ALGORITHM

(71) Applicant: Alphatec Spine, Inc., Carlsbad, CA (US)

(72) Inventors: Kongbin Kang, San Diego, CA (US); Yehyun Suh, Nashville, TN (US); Lin Li, Oceanside, CA (US); Chaochao Zhou, Houston, TX (US)

(21) Appl. No.: 19/447,808

(22) Filed: Jan. 13, 2026

Related U.S. Application Data
(60) Provisional application No. 63/746,214, filed on Jan. 16, 2025.

(51) Int. Cl. A61B 34/20; A61B 17/56; A61B 17/70; A61B 90/00; G06T 7/73; G16H 20/40

(52) U.S. Cl. CPC A61B 34/20; A61B 17/7001; G06T 7/75; G16H 20/40; A61B 2017/564; A61B 2034/2065; A61B 2090/367; A61B 2090/376; G06T 2207/10116; G06T 2207/30012; G06T 2207/30052

(57) ABSTRACT

The present disclosure utilizes images taken of the surgically placed screws to build three-dimensional (3D) reconstructions or models of the screws in order to properly identify and match the screws from the 2D images to the physical screws within a spinal segment. The identification and matching determines the orientation of the screw(s), such as a vertical position (e.g., top or bottom), a handedness (e.g., left or right), an angular position, and a rotational position. Such modeling of the screws within the spinal segment allows a practitioner to, among other things, (i) know which physical screws correlate to the screws within the 2D images, (ii) determine a shape of a rod for placement within the spinal segment based on the orientation of the screw(s), and/or (iii) plan a surgery, such as planning placement of a rod within a spinal segment based on the orientation of the screw(s).

### CROSS REFERENCE TO RELATED MATTER

This application claims priority to U.S. Provisional Application No. 63/746,214, filed Jan. 16, 2025, the entirety of which is incorporated herein by reference.

### TECHNICAL FIELD

This disclosure relates generally to systems and methods for predicting and/or determining an orientation of a plurality of screws from one or more two-dimensional (2D) images.

### SUMMARY

Disclosed are systems, devices, and/or methods of use thereof regarding predicting and/or determining an orientation of a plurality of screws from one or more two-dimensional (2D) images. In various aspects, a method of determining an orientation of at least two screws from at least two, 2D images includes receiving a first set of coordinates for a first screw within the 2D images, with the first set of coordinates corresponding to one or more structural features of a first screw in the 2D images. The method may also include receiving a second set of coordinates for a second screw within the 2D images, with the second set of coordinates corresponding to one or more structural features of the second screw in the 2D images. Additionally, the method may include generating a first, three-dimensional (3D) reconstruction of the first screw and the second screw based on the received coordinates and generating a second 3D reconstruction of the first screw and the second screw based on the received coordinates. Further, the method may include comparing a first 2D projection of the first 3D reconstruction to the 2D images to determine a first loss function and obtaining a minimum value of the first loss function by adjusting the first 3D reconstruction. Still further, the method may include comparing a second 2D projection of the second 3D reconstruction to the 2D images to determine a second loss function and obtaining a minimum value of the second loss function by adjusting the second 3D reconstruction. The method may also include identifying the loss function with a lower minimum value and outputting the 3D reconstruction corresponding to the loss function with the lower minimum value.

In various aspects, a method of determining an orientation of a plurality of screws from a pair of two-dimensional (2D) images includes receiving a first set of coordinates for a first screw within the pair of 2D images. The first set of coordinates may be derived from structural features of the first screw in the pair of 2D images. The pair of 2D images may include an anterior/posterior two-dimensional image of a spinal segment and a lateral two-dimensional image of the spinal segment. The method may also include receiving a second set of coordinates derived from structural features of a second screw within the pair of 2D images. Additionally, the method may include generating a first three-dimensional (3D) reconstruction of the first screw and the second screw from the received coordinates and generating a second 3D reconstruction of the first screw and the second screw from the received coordinates. Further, the method may include projecting the first 3D reconstruction of the first screw and the second screw onto the pair of 2D images, such that the first 3D reconstruction is projected over the plurality of screws within the pair of 2D images. Still further, the method may include projecting the second 3D reconstruction of the first screw and the second screw onto the pair of 2D images, such that the second 3D reconstruction is projected over the plurality of screws within the pair of 2D images. Even further, the method may also include calculating a first loss function for the projection of the first 3D reconstruction based on alignment of the projection and the plurality of screws within the pair of 2D images, and obtaining a minimum value of the first loss function by adjusting the first 3D reconstruction. The method may also include calculating a second loss function for the projection of the second 3D reconstruction based on alignment of the projection and the plurality of screws within the pair of 2D images, and obtaining a minimum value of the second loss function by adjusting the second 3D reconstruction. Additionally, the method may include selecting which minimum value of the first and second loss functions is lower and identifying, based on which loss function is lower, an orientation of the first screw and an orientation of the second screw within the pair of 2D images.

In various aspects, a method of spinal surgical planning includes receiving a pair of two-dimensional (2D) images of a spinal segment containing a plurality of screws, where the pair of 2D images comprises a lateral view of the spinal segment and an anterior/posterior view of the spinal segment. The method may also include receiving a first set of coordinates for a first screw within the plurality of screws, with the first set of coordinates including first screw feature coordinates, and generating a 3D reconstruction of the first screw from the first set of coordinates. Additionally, the method may include receiving a second set of coordinates for a second screw within the pair of 2D images, generating a 3D reconstruction of the second screw from the second set of coordinates, and projecting the 3D reconstruction of the first screw and the 3D reconstruction of the second screw onto the plurality of screws within the pair of 2D images. Further, the method may include determining a first loss function based on alignment between the projections of the 3D reconstruction of the first screw and the 3D reconstruction of the second screw with the plurality of screws. Still further, the method may include optimizing the 3D reconstructions of the first and second screws based on the loss function, identifying the orientation of the first screw and the orientation of the second screw based on the first loss function, and predicting a shape of a rod based on the orientation of the first screw and the orientation of the second screw, the rod for surgical insertion within the spinal segment.

In various aspects, a system for predicting an orientation of a plurality of screws from a pair of two-dimensional (2D) images of a spinal segment containing the plurality of screws may include a display and one or more processors in communication with the display. The one or more processors may include memory capable of storing instructions executable by the one or more processors causing the one or more processors to receive a first set of coordinates for a first screw within the pair of 2D images, with the first set of coordinates comprising first screw structural feature coordinates. The instructions also cause the one or more processors to receive a second set of coordinates for a second screw within the pair of 2D images, with the second set of coordinates comprising second screw structural feature coordinates. Additionally, the instructions also cause the one or more processors to generate a first 3D reconstruction of the first screw and the second screw from the received coordinates and generate a second 3D reconstruction of the first screw and the second screw from the received coordinates. Further, the instructions also cause the one or more processors to project the first 3D reconstruction to the pair of 2D images, project the second 3D reconstruction to the pair of 2D images, determine a first loss function for the first 3D reconstruction based on the projection of the first reconstruction onto the pair of 2D images, and determine a second loss function for the second 3D reconstruction based on the projection of the second reconstruction onto the pair of 2D images. Still further, the instructions cause the one or more processors to adjust the first reconstruction to obtain the minimum loss, adjust the second reconstruction to obtain the minimum loss, and identify the orientation of the first screw and the orientation of the second screw based on a comparison of the first loss function and the second loss function. Even further, the instructions cause the one or more processors to determine rod shape instructions based on the identified orientation of the first screw and the second screw, and send rod shape instructions to a bender, the bender for forming a rod to be placed in the spinal segment.

Other aspects of the disclosed subject matter, as well as features and advantages of various aspects of the disclosed subject matter, should be apparent to those of ordinary skill in the art through consideration of the ensuing description, the accompanying drawings, and the appended claims.

### BRIEF DESCRIPTION OF THE DRAWINGS

In the drawings:

FIG. 1 illustrates a flowchart decision tree of a first method according to the disclosure;

FIG. 2 illustrates a flowchart decision tree of a second method according to the disclosure;

FIG. 3 illustrates a flowchart decision tree of a third method according to the disclosure;

FIG. 4A illustrates a first two-dimensional (2D) image of a spinal segment containing a plurality of screws and FIG. 4B illustrates a second 2D image of the spinal segment and the plurality of screws;

FIG. 5 schematically illustrates a method for generation of a 3D reconstruction of two screws contained within the 2D images;

FIGS. 6A to 6C illustrate example three-dimensional (3D) reconstructions of the plurality of screws from the 2D images of FIGS. 4A and 4B;

FIG. 7A illustrates a visualization of two screws from the 2D images from FIGS. 4A and 4B based on a first prediction and a first loss function and FIG. 7B illustrates a second visualization of the two screws of FIG. 7A after adjusting the generated 3D reconstructions;

FIG. 8A illustrates a comparison of the generated 3D reconstruction of a first screw to the first screw from the plurality of screws in the 2D image of FIG. 4A and FIG. 8B illustrates a comparison of the generated 3D reconstruction of a second screw to the second screw from the plurality of screws in the 2D image of FIG. 4B;

FIGS. 9A and 9B illustrate another pair of 2D images containing a plurality of screws;

FIG. 10 illustrates a gradient of images from the 2D images and a gradient of reconstructions from the 3D reconstructions;

FIGS. 11A through 13 illustrate a first comparison of sets of combinations of generated 3D reconstructions of the plurality of screws and the 2D images containing the plurality of screws to determine which combination is the most accurate;

FIG. 14 illustrates an example table of loss functions calculated for the sets of combinations in order to determine which combination is the most accurate;

FIGS. 15A through 17 illustrate a second comparison of sets of combinations of generated 3D reconstructions of the plurality of screws and the 2D images containing the plurality of screws to identify which combination is the most accurate;

FIGS. 18A through 20 illustrate a third comparison of sets of combinations of generated 3D reconstructions of the plurality of screws and the 2D images containing the plurality of screws to identify which combination is the most accurate; and

FIG. 21 illustrates a system for predicting an orientation of a plurality of screws from a pair of two-dimensional (2D) images of a spinal segment containing the plurality of screws.

### DETAILED DESCRIPTION

During surgical procedures, screws may be positioned or placed within a spinal segment and images of the screws and their placement may be taken. The images are typically two-dimensional (2D) images and may provide an anterior-posterior view of the spinal segment, a lateral view of the spinal segment, or any other view of the spinal segment containing the positioned screws. Accurate matching of pedicle screws in both anteroposterior (AP) and lateral (LAT) images is critical for successful spinal decompression and stabilization during surgery. However, establishing screw correspondence, especially in LAT views, remains a significant clinical challenge. Matching of a single screw from the 2D images to the physical screw within the spinal segment is relatively straightforward. However, matching a plurality of screws from the 2D images to the physical screws within the spinal segment is difficult.

Current matching methods utilize a camera to follow placement of a screw within the spinal segment in real time. Alternative methods require a practitioner to manually identify with a probe surgically placed screws one-by-one while being followed by a camera. However, requirement of a camera within an operating room increases the clutter and number of instruments within the room, making it difficult for practitioners to navigate around the room. Additionally, manual identification of screws is a time- and labor-intensive process that only increases in complexity with an increased number of surgically placed screws.

The present disclosure utilizes dual C-arm images taken of the surgically placed screws to build three-dimensional (3D) reconstructions or models of the screws in order to properly identify and match the screws from the 2D images to the physical screws within a spinal segment. The dual C-arm views are essential for estimating the 3D real-world coordinates of structural features of the screws (e.g., screw tip coordinates and screw tulip coordinates). The present disclosure does not require manual identification of the first set of coordinates or the second set of coordinates.

The identification and matching determines the orientation of the screw(s), such as a vertical position (e.g., top or bottom), a handedness (e.g., left or right), an angular position, and a rotational position. Such modeling of the screws within the spinal segment allows a practitioner to, among other things, (i) know which physical screws correlate to the screws within the 2D images, (ii) determine a shape of a rod for placement within the spinal segment based on the orientation of the screw(s), and (iii) plan a surgery, such as planning placement of a rod within a spinal segment based on the orientation of the screw(s).

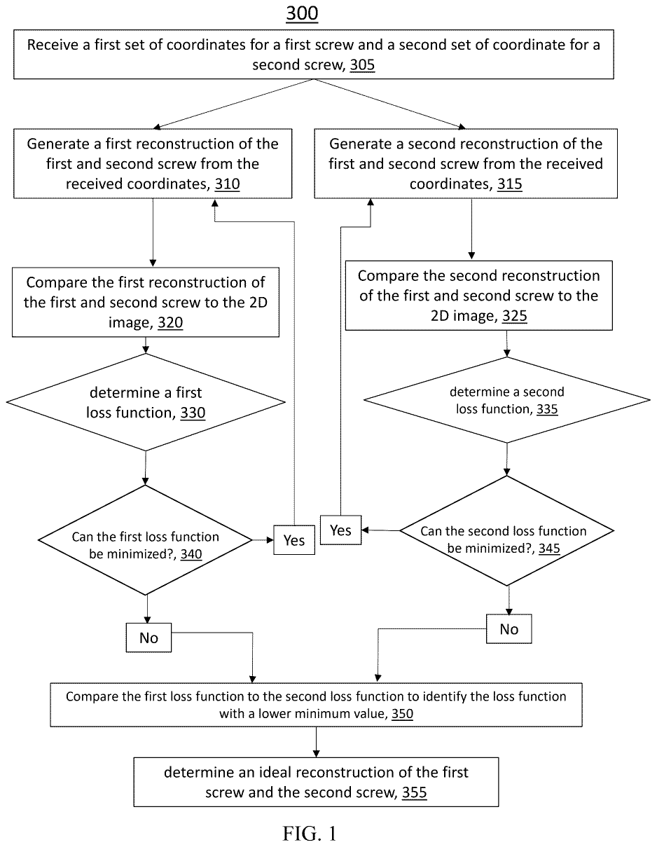

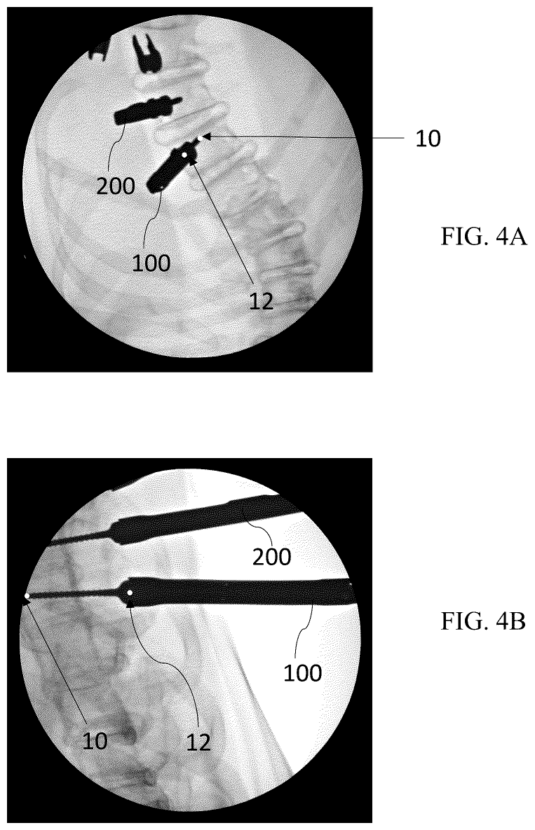

FIG. 1 illustrates a flowchart decision tree of a first method 300 according to the disclosure. The decision tree correlates to a method 300 of determining an orientation of at least two screws from at least two two-dimensional (2D) images and may be executed automatically by a system. At the beginning, the decision tree includes receiving a first set of coordinates for a first screw within the 2D images, 305. The first set of coordinates may correspond to one or more structural features of a first screw in the 2D images. For example, referring briefly to FIGS. 4A and 4B, a first screw 100 and a second screw 200 are pictured in an anterior/posterior (AP) 2D image (FIG. 4A) and in a lateral (LAT) 2D image (FIG. 4B). The structural features of the first screw 100 may include a screw tip 10, a screw head and/or tulip 12, a shank or shaft, or a tower. Computing the coordinates involves annotating landmarks on the 2D images. Projection matrices for the AP view and the LAT view are employed to transform the 3D coordinates into the 2D space.

The method 300 may also include receiving a second set of coordinates for a second screw 200 within the 2D images, 305, with the second set of coordinates corresponding to one or more structural features of the second screw 200 in the 2D images. Similar to the first screw 100, the structural features of the second screw 200 may include a screw tip, a screw head and/or tulip, a shank or shaft, or a tower (not illustrated in FIGS. 4A and 4B).

For example, the 3D coordinates of the structural features (e.g., screw tip and/or screw tulip) may be computed by solving a set of equations using the least square:

$$X ( {p}_{11}^{AP} - {u}_{AP} {p}_{31}^{AP} ) + Y ( {p}_{12}^{AP} - {u}_{AP} {p}_{32}^{AP} ) + Z ( {p}_{13}^{AP} - {u}_{AP} {p}_{33}^{AP} ) = {u}_{AP} {p}_{34}^{AP} - {p}_{14}^{AP}$$

$$X ( {p}_{21}^{AP} - {v}_{AP} {p}_{31}^{AP} ) + Y ( {p}_{22}^{AP} - {v}_{AP} {p}_{32}^{AP} ) + Z ( {p}_{23}^{AP} - {v}_{AP} {p}_{33}^{AP} ) = {v}_{AP} {p}_{34}^{AP} - {p}_{24}^{AP}$$

$$X ( {p}_{11}^{LAT} - {u}_{LAT} {p}_{31}^{LAT} ) + Y ( {p}_{12}^{LAT} - {u}_{LAT} {p}_{32}^{LAT} ) + Z ( {p}_{13}^{LAT} - {u}_{LAT} {p}_{33}^{LAT} ) = {u}_{LAT} {p}_{34}^{LAT} - {p}_{14}^{LAT}$$

$$X ( {p}_{21}^{LAT} - {v}_{LAT} {p}_{31}^{LAT} ) + Y ( {p}_{22}^{LAT} - {v}_{LAT} {p}_{32}^{LAT} ) + Z ( {p}_{23}^{LAT} - {v}_{LAT} {p}_{33}^{LAT} ) = {v}_{LAT} {p}_{34}^{LAT} - {p}_{24}^{LAT}$$

where (U_{AP}, V_{AP}) and (U_{LAT}, V_{LAT}) represent the screw tip and screw tulip coordinates of the screws on each 2D image plane. The elements p^{AP}ᵢⱼ and p^{LAT}ᵢⱼ are part of the projection matrices that map the 3D coordinates into the 2D space. The 3D coordinates of the structural features may also be computed according to other computational methods.

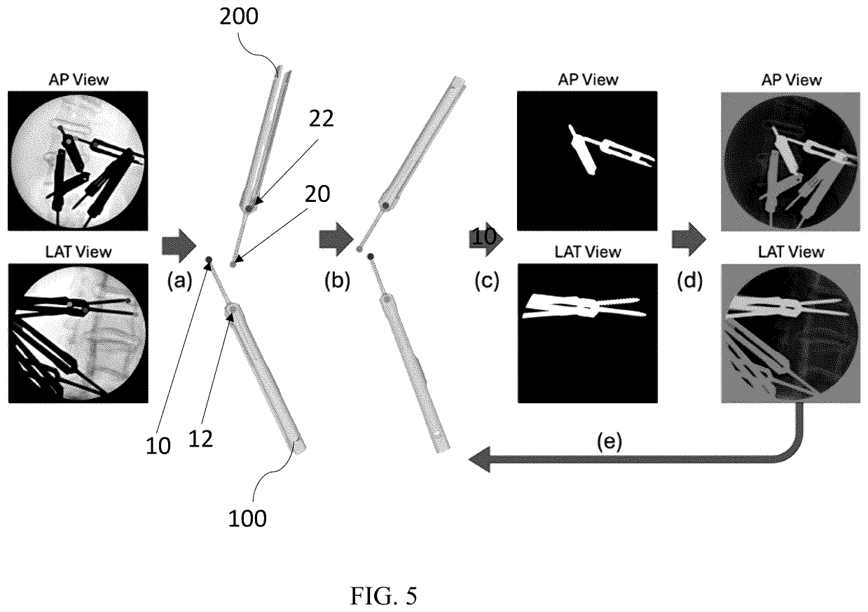

After estimation of the 3D coordinates of the screw structural features, a vector from the screw tulip to the screw tip may be computed as:

$$V = C - T = ( {X}_{C} - {X}_{T} , {Y}_{C} - {Y}_{T} , {Z}_{C} - {Z}_{T} )$$

where C=(X_{c}, Y_{c}, Z_{c}) is the center of the tulip and T=(X_{T}, Y_{T}, Z_{T}) is the tip of the screw. The vector can also be computed according to other methods. This vector V can be used to align the generated models of the screws in 3D space by positioning the center and the tip at the corresponding real-world coordinates. For example, FIG. 5 schematically illustrates generation of 3D reconstruction of the screws based on the coordinates and vectors calculated.

Additionally, the method 300 may include generating a first three-dimensional (3D) reconstruction of the first screw 100 and the second screw 200 based on the received coordinates, 310, and generating a second 3D reconstruction of the first screw 100 and the second screw 200 based on the received coordinates, 315. The 3D coordinates may be calculated from the 2D coordinates in the AP and LAT views. The generated 3D model for the first screw 100 and the second screw 200 may be aligned with each other. The generated 3D model for the first screw 100 and the second screw 200 may also be rotated and translated to find the best fit between the 3D reconstruction of the first screw 100 and the first screw 100 contained within the 2D images.

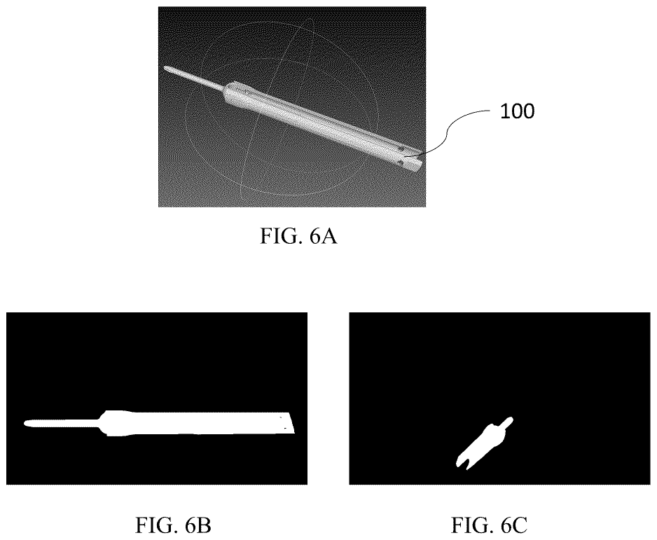

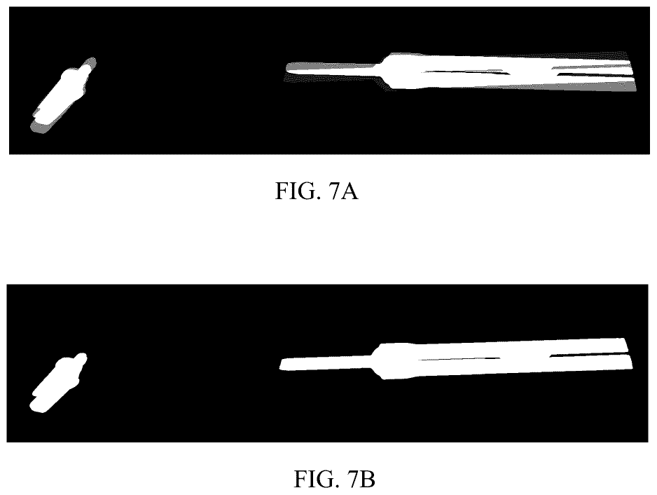

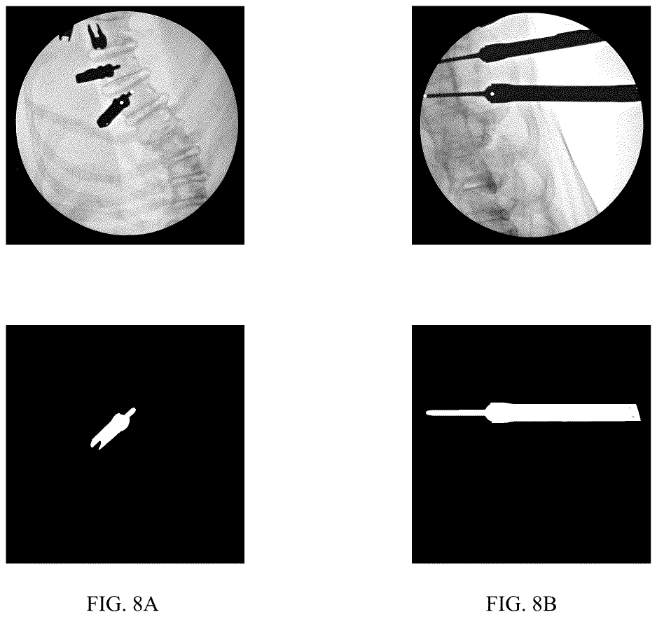

In one embodiment, the 3D reconstructions of the first screw 100 and the second screw 200 may be projected into the AP and LAT views to compare the reconstruction to the 2D image, 320, 325, such as at (c) in FIG. 5, to create 2D projections of the first screw 100 and the second screw 200. This is also illustrated in FIGS. 6A through 6C, where FIG. 6A shows a modeled 3D reconstruction of a screw and FIGS. 6B and 6C illustrate 2D projections of the modeled screw. FIG. 8A shows the AP 2D image containing a first screw 100 on top with the 2D projection of the first screw 100 on the bottom. Similarly, FIG. 8B shows the LAT 2D image containing the first screw 100 on top with the 2D projection of the first screw 100 on the bottom.

Further, the method 300 may include comparing a first 2D projection of the first 3D reconstruction to the 2D images to determine a first loss function, 330. For example, as shown in (d) of FIG. 5, the first 2D projection is overlaid onto both the AP and LAT 2D images to determine a fit or match between the first 2D projection and the screws within the AP and LAT 2D images. The overlays may provide a 2D binary map in both the AP and LAT views.

The 2D binary map is created from projecting meshes and covering the entire projection region. For example, let X₁, X₂, and X₃ represent the 2D coordinates of the three vertices projected from the 3D models onto the 2D image plane. These coordinates form a triangle A (X₁, X₂, X₃) and the binary mask Iₚᵣₒⱼ(x,y) is defined as:

$${I}_{proj} ( x , y ) = \{ \begin{matrix}1 , & if ( x , y ) is inside Δ ( X 1 , X 2 , X 3 ) \\ 0 , & otherwise\end{matrix}$$

Here, for each triangle in the 3D model, the corresponding 2D projection forms a triangle on the 2D image. The pixels that lie within this triangle are assigned a value of 1 in the binary mask, representing the projected region of the screw. Then, background removal is applied by multiplying the projected image Iₚᵣₒⱼ (x,y) with a predetermined background mask M_{bg}(x,y), which eliminates irrelevant regions. The final binary map I_{final}(x,y) is computed as:

$${I}_{final} ( x , y ) = {I}_{proj} ( x , y ) \cdot {M}_{b g} ( x , y )$$

The binary map may also be created by other suitable computation methods.

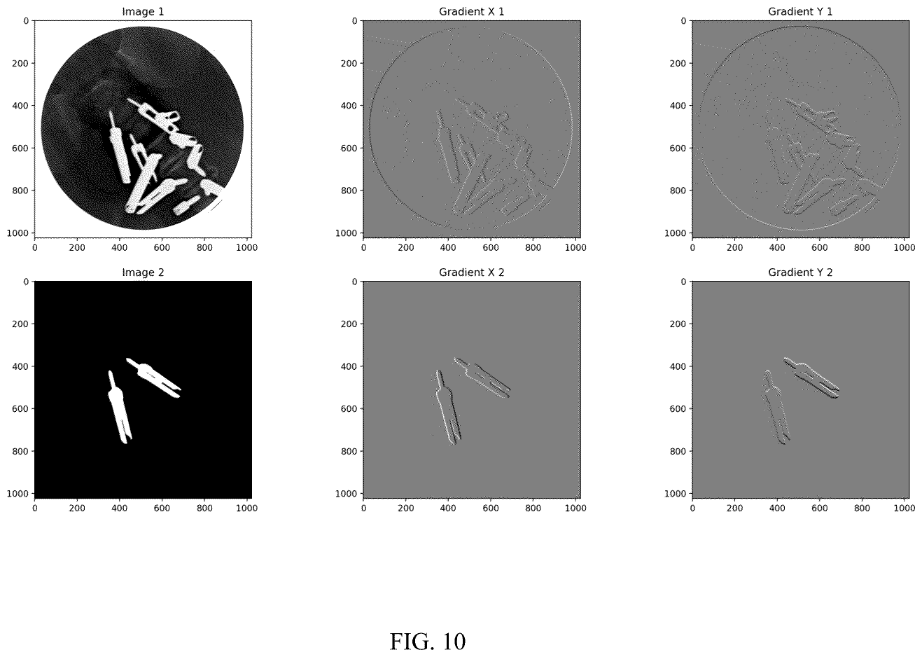

Similarity between the projection and the ground truth image (e.g., the 2D AP and/or LAT images) may be evaluated according to any suitable method. In one method, similarity between the projection and ground truth may be evaluated using the Gradient Correlation Loss, which measures alignment of the image gradients, such as shown in (d) of FIG. 5 and FIG. 10. Accuracy of this fit or match may be correlated to the loss function computed. Given the projected image Iₚᵣₒⱼ and the real image Iᵣₑₐₗ, the gradients in the x- and y-directions are:

$$∇ {I}_{proj} = ( \frac{\partial {I}_{proj}}{\partial x} , \frac{\partial {I}_{proj}}{\partial y} ) , ∇ {I}_{r e a l} = ( \frac{\partial {I}_{real}}{\partial x} , \frac{\partial {I}_{real}}{\partial y} )$$

The Gradient Correlation Loss L_{grad} can be simplified as:

$${L}_{grad} = - \frac{{\sum }_{i , j} ∇ {I}_{p r o j} ( i , j ) \cdot ∇ {I}_{real} ( i , j )}{\sqrt{{\sum }_{i , j} {‖ ∇ {I}_{proj} ( i , j ) ‖}^{2}} \cdot \sqrt{{\sum }_{i , j} {‖ ∇ {I}_{real} ( i , j ) ‖}^{2}}}$$

Here, ∇Iₚᵣₒⱼ(i, j) and ∇Iᵣₑₐₗ(i, j) represent the gradients of the projected and real images at each pixel (i, j) and ∥VIₚᵣₒⱼ(i, j)∥² and |∇Iᵣₑₐₗ(i, j)∥² is the squared magnitude of the gradient in each image. The numerator computes the dot products of the gradients, while the denominator normalizes them by their magnitudes, ensuring scale invariance. FIG. 10 illustrates image gradients produced for both the AP and LAT 2D image views as well as gradients produced for the projected image of the screw.

Based on this overlay and the loss function, the method 300 includes obtaining a minimum value of the first loss function by adjusting the first 3D reconstruction. As shown in FIG. 1, the method 300 may include determining if the calculated loss function can be minimized, 340. If the calculated loss function can be minimized, the process is repeated (i.e., returning to 310) until the loss function cannot be minimized. This loss is minimized when the gradients of the two images are aligned, minimizing to −1 when they are perfectly aligned. This allows optimization processes to have more accurate pose estimation of the screws making correlation between the projection and the ground truth more similar to each other.

The optimization seeks to minimize the total loss of L_{grad} which is the combination of the loss from AP and LAT views:

$${L}_{grad} = {L}_{A P} + {L}_{L A T}$$

where L_{AP} and L_{LAT} are the loss from the AP and LAT views. The optimization is formulated as:

$${θ}^{*} = arg {min}_{θ} {L}_{total} ( θ )$$

where θ* represents the optimal screw pose parameters (translation and rotation) that minimize the total loss and align the projection of the modeled screws with the real screw. For optimization, differential evolution may be used. Differential evolution may be effective for global optimization over continuous spaces, and in exploring large search spaces and avoiding local minima. It iteratively adjusts the pose of the screws, seeking for the best alignment as shown in (e) of FIG. 5.

Still further, the method 300 may include comparing a second 2D projection of the second 3D reconstruction to the 2D images, 325, to determine a second loss function, 335, and obtaining a minimum value of the second loss function by adjusting the second 3D reconstruction (the right side of the flow chart in FIG. 1). The second loss function may be calculated similar to the first loss function. If the second loss function can be further minimized, 345, the second loss function can similarly be iteratively minimized through the Gradient Correlation Loss described.

In one embodiment, when the first loss function and the second loss function cannot be further minimized, the method 300 may also include comparing the loss functions and identifying the loss function with a lower minimum value, 350, and outputting the 3D reconstruction corresponding to the loss function with the lower minimum value. That is, the first loss function (after minimization) is compared to the second loss function (after minimization). The 3D reconstruction corresponding to the loss function with the lower minimum value provides a more accurate fit between the 3D reconstruction of the screws and the screws contained within the AP and LAT 2D images, 355. In this way, refinement and optimization of the loss functions predicts which of the first and second screws of the 3D reconstruction match the first screw 100 and the second screw 200 in the AP and LAT 2D images. This matching both identifies the first screw 100 and the second screw 200 in the AP and LAT 2D images, as well as predicts or identifies the orientation of the first screw 100 relative to the second screw 200 (e.g., top or bottom, left or right, and the angular or rotational positioning).

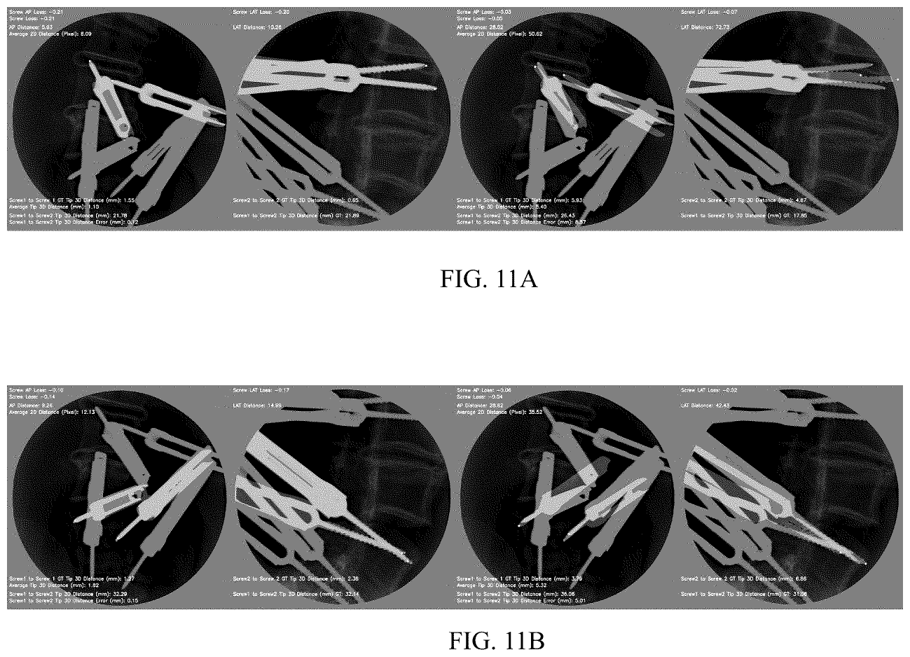

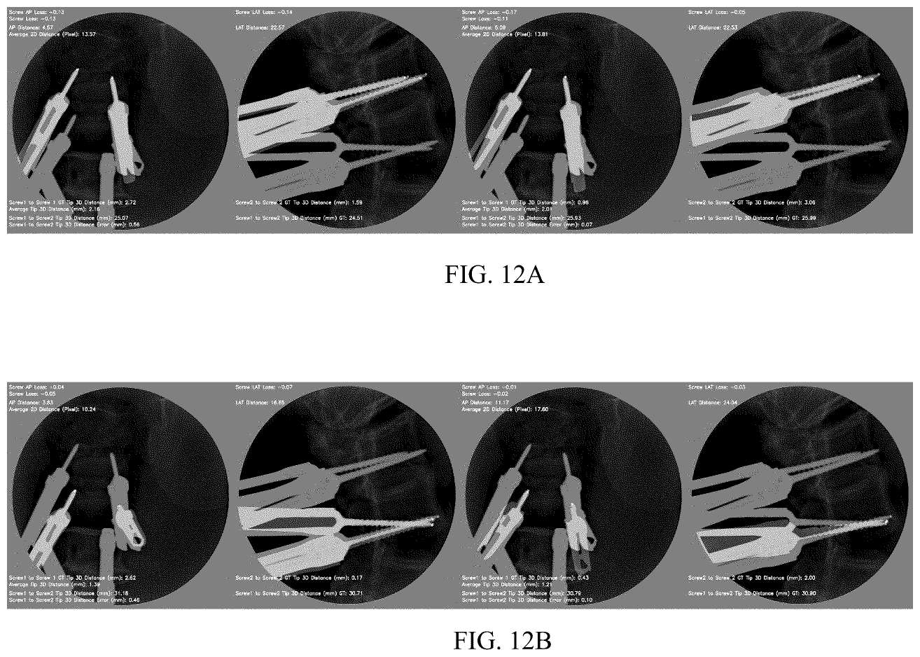

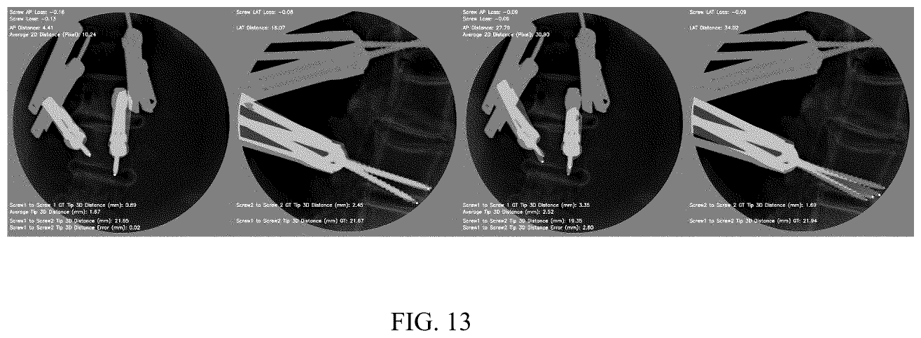

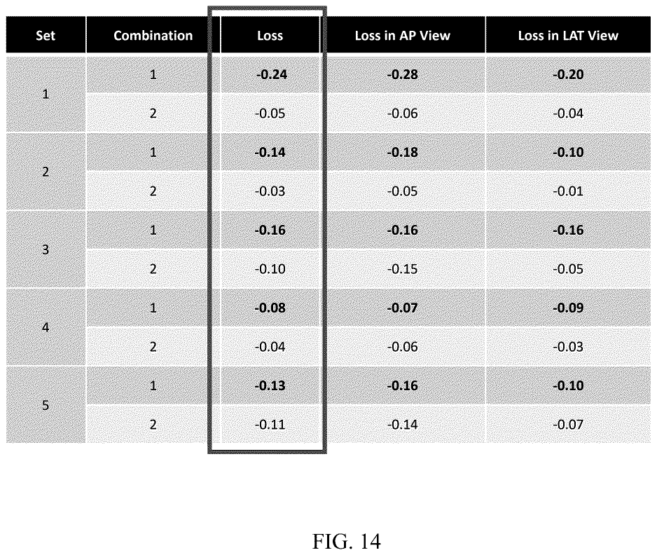

For example, FIGS. 11A through 13 illustrate projections of 2D projections of the 3D reconstructions of the first screw 100 and the second screw 200 onto the AP and LAT 2D images and direct comparisons of these projections to optimize and refine the fit. FIG. 14 shows an example comparison of the loss functions for each projection to identify which projection has the minimal loss function, which represents the most accurate fit. The projection with the minimal loss function allows a practitioner to identify which is the first screw 100 and which is the second screw 200, and their orientations relative to each other, between the AP and LAT 2D images.

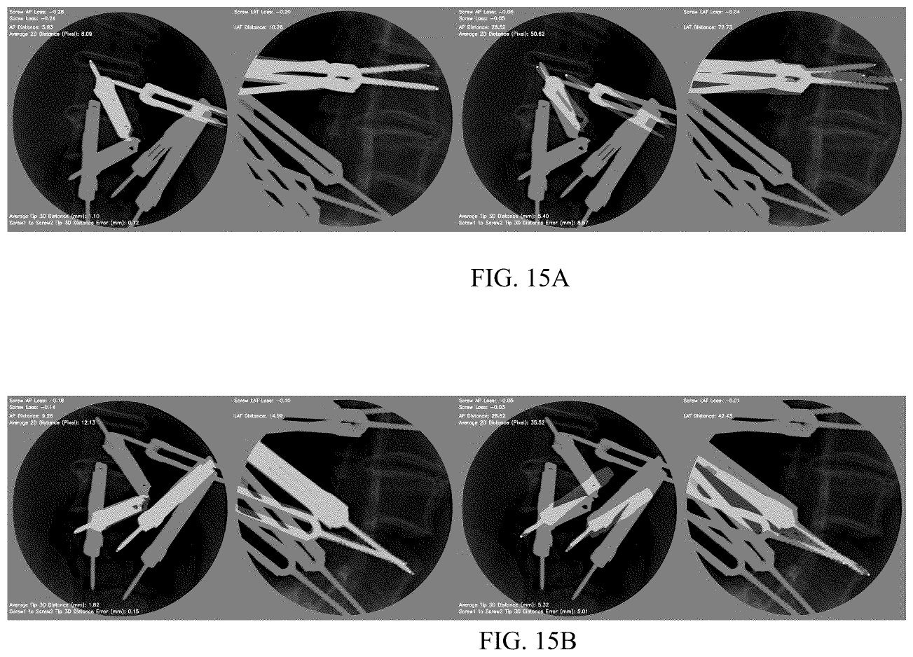

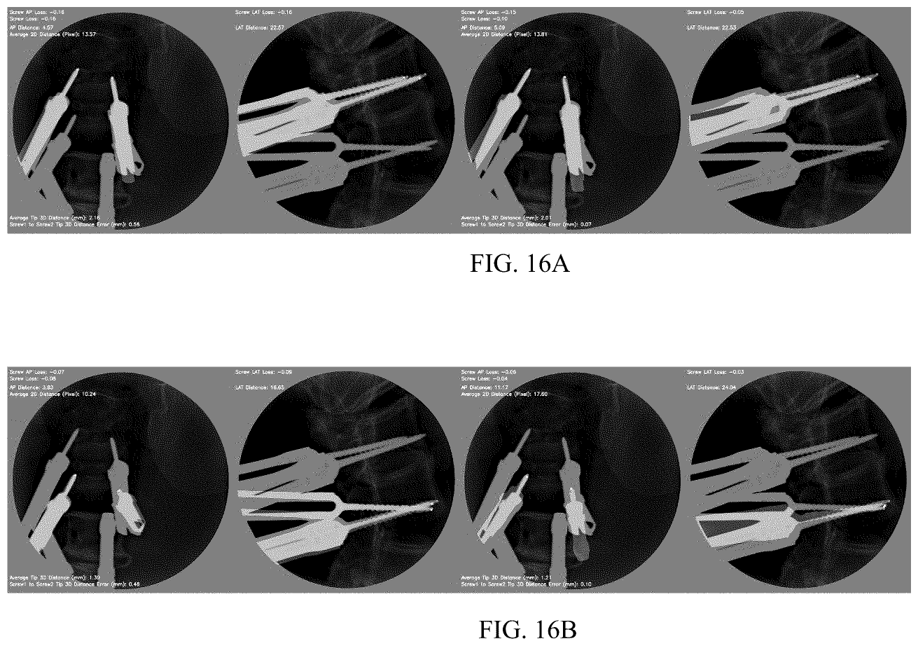

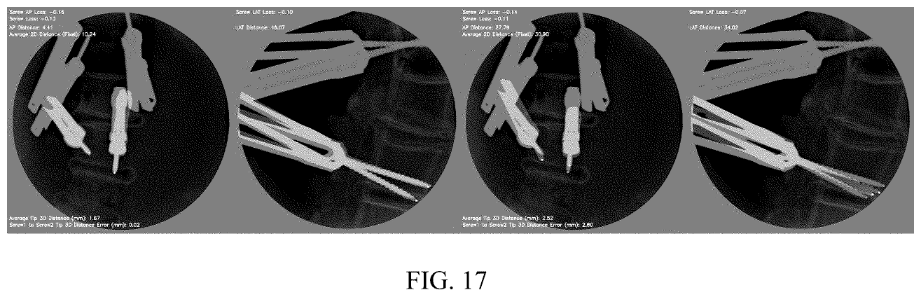

FIGS. 15A through 17 illustrate a second group of comparisons of 2D projections of the 3D reconstructions of the first screw 100 and the second screw 200 onto the AP and LAT 2D images. As before, these comparisons illustrate which 2D projections of the 3D reconstructions of the first screw 100 and the second screw 200 best match or fit onto the screws within the AP and LAT 2D images. Also as before, the 2D projection with the lowest loss function will represent the best fit between the 2D projections of the first screw and the second screw and the first screw 100 and the second screw 200 within the AP and LAT 2D images.

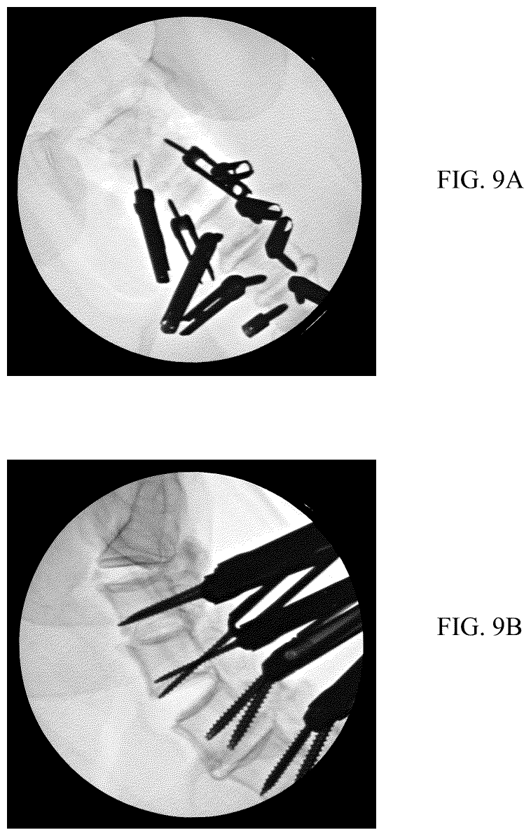

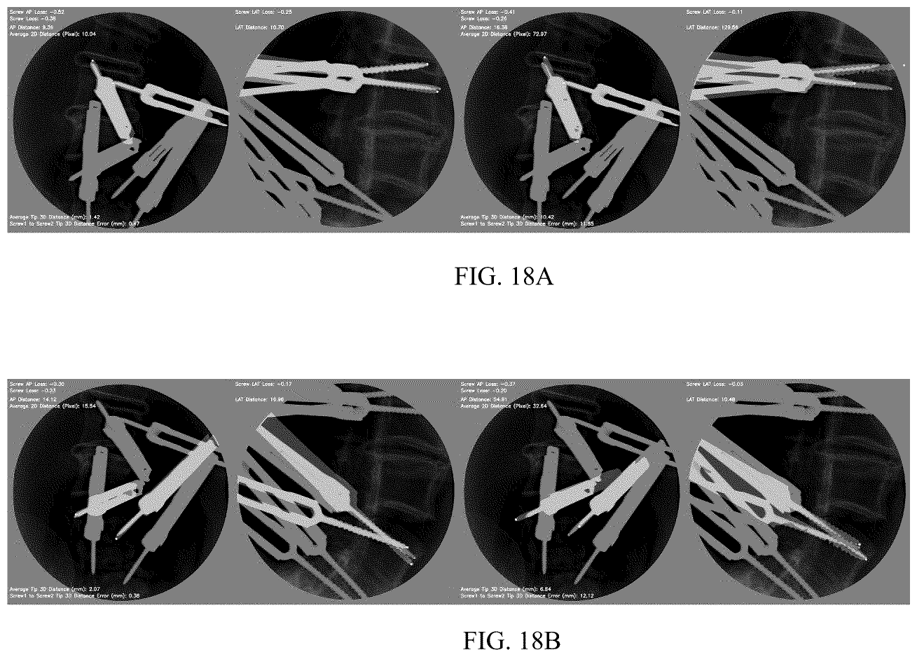

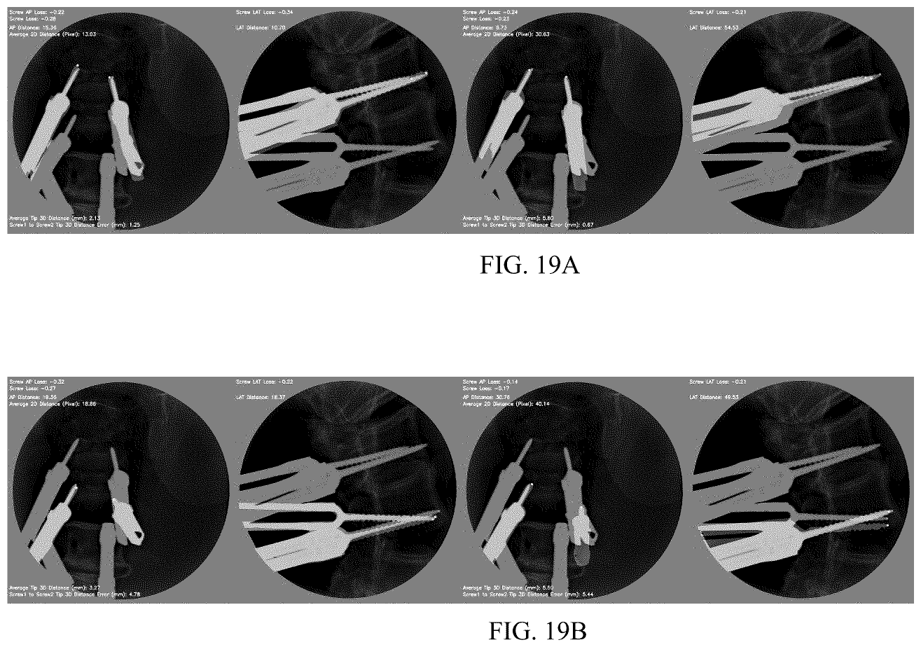

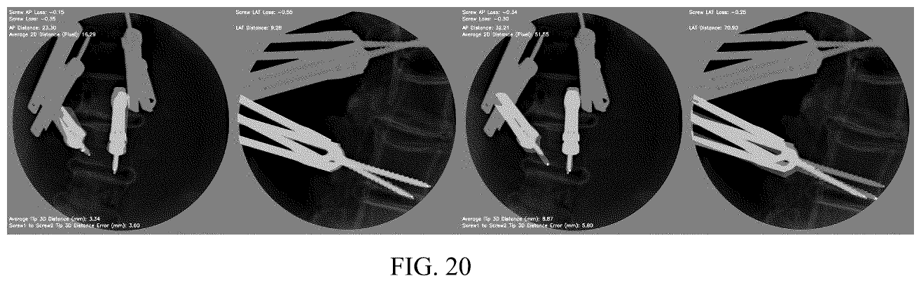

FIGS. 18A through 20 represent a third group of comparisons to find the 2D projection with the best fit (i.e., the lowest loss function). Once the orientation of the screws has been identified or predicted, the method may be repeated for additional screws contained within the 2D images (e.g., a third screw, a fourth screw, etc.). FIGS. 9A and 9B illustrate one example of 2D images containing a plurality of screws whose orientation can each be identified or predicted within a spinal segment. Once the orientation of the plurality of screws has been determined, a practitioner can predict a shape of a rod to be placed in the spinal segment where the screws are. Without knowing the orientations of the screws, the rod shape may not be precise enough for surgical implantation within the spinal segment.

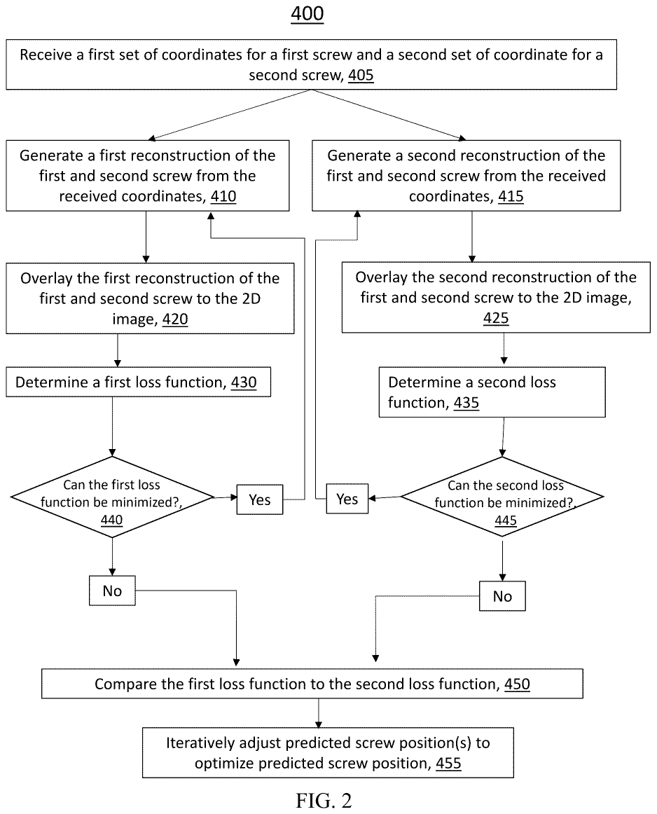

FIG. 2 illustrates a flowchart decision tree of a second method 400 according to the disclosure. The decision tree correlates to a method 400 of determining an orientation of at least two screws from at least two two-dimensional (2D) images and may be executed by a system. At the beginning, the decision tree includes receiving a first set of coordinates for a first screw within the pair of 2D images, 405. Similar to the method 300 of FIG. 1, the first set of coordinates may be derived from structural features of the first screw (e.g., screw tip coordinates, screw tulip coordinates, screw head coordinates, screw shaft coordinates, etc.) in the pair of 2D images. As before, the pair of 2D images may include an anterior/posterior (AP) two-dimensional image of a spinal segment and a lateral (LAT) two-dimensional image of the spinal segment. The AP and LAT images may be C-arm images that are oriented at an angle relative to each other, such as an angle greater than about 30 degrees (e.g., 32, 34, 36, 40, 42, 45, 50, 55, 60, 65, 70, 75, 80, 85, 90 degrees, or an angle within a range defined by any two of the foregoing values, etc.)

The method 400 may also include receiving a second set of coordinates derived from structural features of a second screw within the 2D image, 405. Additionally, the method 400 may include generating a first three-dimensional (3D) reconstruction of the first screw and the second screw from the received coordinates, 410, and generating a second 3D reconstruction of the first screw and the second screw from the received coordinates, 415.

Further, the method 400 may include projecting (e.g., visually overlaying) the first 3D reconstruction of the first screw and the second screw onto the pair of 2D images, such that the first 3D reconstruction is projected over the plurality of screws within the pair of 2D images, 420. Still further, the method 400 may include projecting (e.g., visually overlaying) the second 3D reconstruction of the first screw and the second screw onto the pair of 2D images, such that the second 3D reconstruction is projected over the plurality of screws within the pair of 2D images, 425. The method 400 may also include calculating a first loss function for the projection of the first 3D reconstruction based on alignment of the projection and the plurality of screws within the pair of 2D images, 430, and obtaining a minimum value of the first loss function by adjusting the first 3D reconstruction. As before, the first loss function may be calculated through a gradient of the 2D images and gradients of the 2D projections of the first screw and the second screw. If the first loss function can be minimized, 440, the first loss function will be iteratively minimized as described to obtain the minimal value.

The method 400 may also include calculating a second loss function for the projection of the second 3D reconstruction based on alignment of the projection and the plurality of screws within the pair of 2D images, 435, and obtaining a minimum value of the second loss function by adjusting the second 3D reconstruction (the right side of the flow chart of FIG. 2). If the second loss function can be minimized, 445, the second loss function will be iteratively minimized as described to obtain the minimal value. Additionally, the method 400 may include comparing and selecting which minimum value of the first and second loss functions is lower, 450, and identifying, 455, based on which loss function is lower, an orientation of the first screw and an orientation of the second screw within the pair of 2D images.

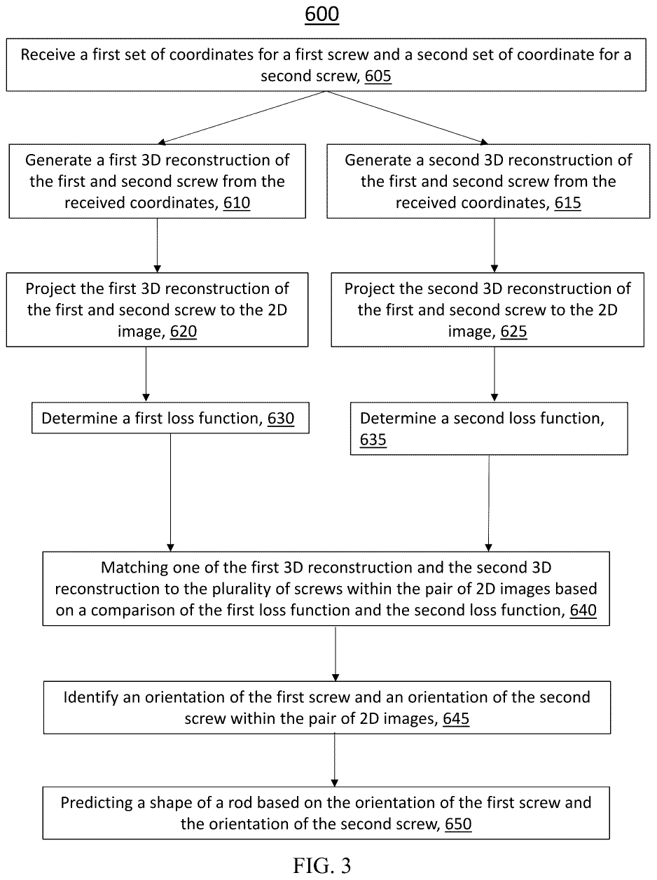

FIG. 3 illustrates a flowchart decision tree of a third method 600 according to the disclosure. The decision tree may correlate to a method 600 of surgical planning, such as predicting a shape for a rod to be surgical implanted within a spinal segment. As with the methods 300, 400 of FIGS. 1 and 2, the method 600 of FIG. 3 utilizes a pair of C-arm 2D images to generate screw models to thereby properly identify and orient screws within the pair of C-arm 2D images. As before, the method 600 may include receiving a pair of two-dimensional (2D) images of a spinal segment containing a plurality of screws, the pair of 2D images comprising a lateral (LAT) view of the spinal segment and an anterior/posterior (AP) view of the spinal segment. FIGS. 4A to 4B and FIGS. 9A and 9B illustrate example 2D images containing a plurality of screws within a spinal segment.

The method 600 further includes receiving a first set of coordinates for a first screw within the plurality of screws, the first set of coordinates including first screw feature coordinates, 605, and generating a 3D reconstruction of the first screw from the first set of coordinates, 610. FIG. 5 illustrates the generation of the 3D reconstruction of the first screw 100 from the pair of 2D images. FIG. 6A also illustrates the 3D reconstruction of the first screw 100. The method 600 also includes receiving a second set of coordinates for a second screw within the pair of 2D images, 605, and generating a 3D reconstruction of the second screw from the second set of coordinates, 615.

Additionally, the method 600 may include projecting the 3D reconstruction of the first screw and the 3D reconstruction of the second screw onto the plurality of screws within the pair of 2D images, 620, 625, as illustrated in FIGS. 11A to 13, FIGS. 15A to 17, and FIGS. 18A to 20. The method 600 may include determining a first loss function based on alignment between the projections of the 3D reconstruction of the first screw and the 3D reconstruction of the second screw with the plurality of screws, 630. The method 600 may include determining a second loss function based on alignment between the projections of the 3D reconstruction of the first screw and the 3D reconstruction of the second screw with the plurality of screws, 635. As before, the first loss function and/or second loss function may be calculated using the Gradient Correlation Loss described. FIG. 10 illustrates examples of gradients for both the AP and LAT 2D images and reconstructions of the screws. Additionally, and/or alternatively, a clinician may determine the better alignment through visual comparison of the gradients and overlays of the AP, LAT 2D images, and reconstructions of the screws.

The method 600 may also include optimizing the 3D reconstructions of the first and second screws based on the loss function, as described. Additionally, the method 600 may include matching one of the first 3D reconstruction and the second 3D reconstruction to the plurality of screw within the pair of 2D images based on a comparison of the first loss function and the second loss function, 640, and identifying the orientation of the first screw and the orientation of the second screw based on a minimized loss function, 645. Based on the identified orientation, the method also includes predicting a shape of a rod based on the orientation of the first screw and the orientation of the second screw, the rod for surgical insertion within the spinal segment, 650.

As with the methods 300, 400 of FIGS. 1 and 2, the method 600 of FIG. 3 may be iteratively repeated for a third screw, a fourth screw, a fifth screw, etc. until each screw contained within the pair of 2D images has been identified and oriented relative to each other. For each screw, a loss function will be calculated and, if possible, optimized to its minimal value. The loss function with the most minimal value will correlate to the 3D reconstruction that most accurately aligns the screws within the 2D images when projected onto the 2D images, thereby predicting and identifying the screws (e.g., their orientation relative to each other) within the 2D images.

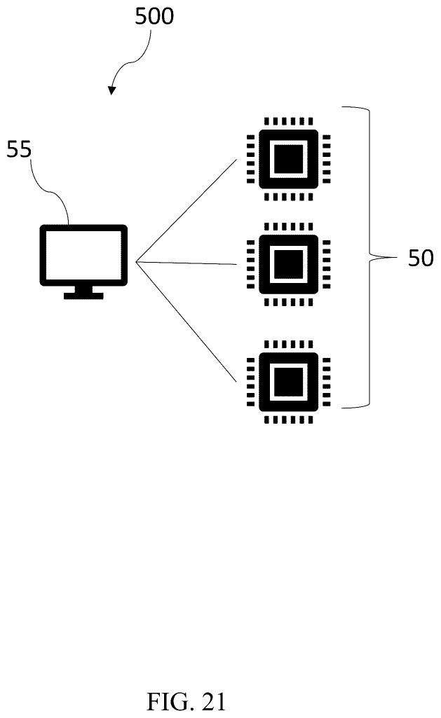

FIG. 21 illustrates a system 500 for predicting an orientation of a plurality of screws from a pair of two-dimensional (2D) images of a spinal segment containing the plurality of screws. The system 500 may include a display 55 and one or more processors 50 in communication with the display 55. The one or more processors 50 may include memory capable of storing instructions executable by the one or more processors 50 causing the one or more processors 50 to receive a first set of coordinates for a first screw within the pair of 2D images, with the first set of coordinates comprising first screw structural feature coordinates. The instructions also cause the one or more processors 50 to receive a second set of coordinates for a second screw within the pair of 2D images, with the second set of coordinates comprising second screw structural feature coordinates. Additionally, the instructions also cause the one or more processors 50 to generate a first 3D reconstruction of the first screw and the second screw from the received coordinates and generate a second 3D reconstruction of the first screw and the second screw from the received coordinates.

Further, the instructions cause the one or more processors 50 to project the first 3D reconstruction to the pair of 2D images, project the second 3D reconstruction to the pair of 2D images, determine a first loss function for the first 3D reconstruction based on the projection of the first reconstruction onto the pair of 2D images, and determine a second loss function for the second 3D reconstruction based on the projection of the second reconstruction onto the pair of 2D images. Still further, the instructions cause the one or more processors 50 to adjust the first reconstruction to obtain the minimum loss, adjust the second reconstruction to obtain the minimum loss, and identify the orientation of the first screw and the orientation of the second screw based on a comparison of the first loss function and the second loss function. Even further, the instructions cause the one or more processors 50 to determine rod shape instructions based on the identified orientation of the first screw and the second screw, and send rod shape instructions to a bender, the bender for forming a rod to be placed in the spinal segment.

The display 55 may visually overlay the 3D reconstruction of the first screw and the 3D reconstruction of the second screw on the pair of 2D images. The instructions may further cause the one or more processors 50 to display the first 3D reconstruction over the pair of 2D images. Additionally, the instructions may cause the one or more processors 50 to display a rod shape corresponding to the orientation of the first screw and the orientation of the second screw. Still further, the instructions may cause the one or more processors 50 to detect a change in orientation of the first screw, generate an updated 3D reconstruction of the first screw and the second screw based on the change detected, project the updated 3D reconstruction of the first screw and the second screw onto the pair of 2D images, calculate an updated loss function based on projecting the updated 3D reconstruction onto the pair of 2D images, and determine an updated rod shape based on the change in orientation of the first screw. The change in orientation of the first screw may correlate to a position of a vertebrae within the spinal segment. This updated 3D reconstruction and rod shape may be displayed on the display 55.

### EMBODIMENTS

The following embodiments are provided as examples only of specific configurations, materials, arrangements, etc. contemplated by the authors of this disclosure: Embodiment 1. A method of determining an orientation of at least two screws from at least two, two-dimensional (2D) images, the method comprising:
receiving a first set of coordinates for a first screw within the 2D images, the first set of coordinates corresponding to one or more structural features of a first screw in the 2D images;
receiving a second set of coordinates for a second screw within the 2D images, the second set of coordinates corresponding to one or more structural features of the second screw in the 2D images;
generating a first 3D reconstruction of the first screw and the second screw based on the received first coordinates;
generating a second 3D reconstruction of the first screw and the second screw based on the received second coordinates;
comparing a first 2D projection of the first 3D reconstruction to the 2D images to determine a first loss function;
obtaining a minimum value of the first loss function by adjusting the first 3D reconstruction;
comparing a second 2D projection of the second 3D reconstruction to the 2D images to determine a second loss function;
obtaining a minimum value of the second loss function by adjusting the second 3D reconstruction;
comparing the minimum value of the first loss function to the minimum value of the second loss function to identify the loss function with a lower minimum value;
outputting the 3D reconstruction corresponding to the loss function with the lower minimum value.

Embodiment 2. The method of embodiment 1, wherein adjusting the first 3D reconstruction comprises iteratively adjusting the first 3D reconstruction to maximize alignment between the first 2D projection and the at least two 2D images, and wherein adjusting the second 3D reconstruction comprises iteratively adjusting the second 3D reconstruction to maximize alignment between the second 2D projection and the at least two 2D images.

Embodiment 3. The method of embodiment 1 or 2, wherein the method is repeated for a third screw and a fourth screw.

Embodiment 4. The method of embodiment 3, further comprising determining a rod shape based on the 3D reconstructions of the first, second, third, and fourth screws.

Embodiment 5. The method of embodiment 4, further comprising sending instructions correlated to the rod shape to a bender for forming a rod having the rod shape.

Embodiment 6. The method of any one of embodiments 1 through 5, wherein predicting the orientation of the first screw and the orientation of the second screw comprises predicting (i) a handedness, (ii) a vertical position, (iii) an angular position, and/or (iv) a rotational position for both the first screw and the second screw.

Embodiment 7. The method of any one of embodiments 1 through 6, wherein comparing the first 2D projection to the 2D images comprises visually overlaying the first 2D projection on each of the 2D images, such that the first 2D projection aligns over at least one screw within the 2D images.

Embodiment 8. The method of any one of embodiments 1 through 7, wherein generating a first 3D reconstruction of the first screw and the second screw from the received coordinates comprises:
receiving x-ray intrinsic parameters and relative extrinsic parameters between two views;
triangulating the first screw features coordinates;
triangulating the second screw features coordinates; and
generating an initial 3D reconstruction of the first screw and the second screw.

Embodiment 9. The method of any one of embodiments 1 through 8, wherein the one or more structural features comprise a screw tip, a screw shaft, a screw head, and/or a tulip.

Embodiment 10. The method of any one of embodiments 1 through 9, wherein the at least two 2D images are oriented at an angle relative to each other, the angle being greater than 10 degrees, greater than 20 degrees, greater than 30 degrees, greater than 40 degrees, greater than 50 degrees, greater than 60 degrees, greater than 70 degrees, or greater than 80 degrees.

Embodiment 11. The method of any one of embodiments 1 through 10, wherein the at least two 2D images are oriented at an angle relative to each other, the angle being less than about 170 degrees, less than about 160 degrees, less than about 150 degrees, less than about 140 degrees, less than about 130 degrees, less than about 120 degrees, less than about 110 degrees, or less than about 100 degrees.

Embodiment 12. The method of any one of embodiments 1 through 9, wherein the at least two 2D images are oriented at an angle relative to each other, the angle being from about 60 degrees to about 120 degrees, from about 70 degrees to about 110 degrees, or from about 80 degrees to about 100 degrees, from about 85 degrees to about 95 degrees, or about 90 degrees.

Embodiment 13. The method of any one of embodiments 1 through 12, wherein the at least two 2D images comprises a lateral image and an anterior/posterior image of a spinal segment of a patient.

Embodiment 14. The method of any one of embodiments 1 through 13, wherein adjusting the first 3D reconstruction comprises iteratively rotating the first 3D reconstruction to maximize alignment between the first 2D projection and the first 3D reconstruction, and wherein adjusting the second 3D reconstruction comprises iteratively rotating the second 3D reconstruction to maximize alignment between the second 2D projection and the second 3D reconstruction.

Embodiment 15. A method of determining an orientation of a plurality of screws from a pair of 2D images, the method comprising:
receiving a first set of coordinates for a first screw within the pair of 2D images,
   the first set of coordinates derived from structural features of the first screw in the pair of 2D images, and
   the pair of 2D images comprising an anterior/posterior two-dimensional image of a spinal segment and a lateral two-dimensional image of the spinal segment;
receiving a second set of coordinates derived from structural features of a second screw within the 2D image;
generating a first three-dimensional (3D) reconstruction of the first screw and the second screw from the received first coordinates;
generating a second 3D reconstruction of the first screw and the second screw from the received second coordinates;
projecting the first 3D reconstruction of the first screw and the second screw onto the pair of 2D images, such that the first 3D reconstruction is projected over the plurality of screws within the pair of 2D images;
projecting the second 3D reconstruction of the first screw and the second screw onto the pair of 2D images, such that the second 3D reconstruction is projected over the plurality of screws within the pair of 2D images;
calculating a first loss function for the projection of the first 3D reconstruction based on alignment of the projection and the plurality of screws within the pair of 2D images;
obtaining a minimum value of the first loss function by adjusting the first 3D reconstruction;
calculating a second loss function for the projection of the second 3D reconstruction based on alignment of the projection and the plurality of screws within the pair of 2D images;
obtaining a minimum value of the second loss function by adjusting the second 3D reconstruction;
comparing the minimum value of the first loss function and the minimum value of the second loss function and selecting the 3D reconstruction with the lower loss function; and
identifying, based on the 3D reconstruction with the lower loss function, an orientation of the first screw and an orientation of the second screw within the pair of 2D images.

Embodiment 16. The method of embodiment 15, wherein generating a first three-dimensional (3D) reconstruction of the first screw and the second screw from the received coordinates comprises generating a gradient of 2D images of the first screw and the second screw.

Embodiment 17. The method of embodiment 16, wherein calculating a first loss function comprises:
measuring a similarity of the gradient of 2D images of the first screw and the second screw with a gradient of the pair of 2D images; and
computing a similarity score between both gradients for consistency.

Embodiment 18. The method of any one of embodiments 15 through 17, wherein identifying the orientation of the first screw and the orientation of the second screw within the pair of 2D images comprises visually matching a 3D reconstruction of the first screw with the first screw within the pair of 2D images and visually confirming a 3D reconstruction of the second screw with the second screw within the pair of 2D images.

Embodiment 19. The method of any one of embodiments 15 through 18, wherein the method does not require manual identification of the first set of coordinates or the second set of coordinates.

Embodiment 20. The method of any one of embodiments 15 through 19, wherein the pair of 2D images are oriented at an angle relative to each other, the angle being from about 60 degrees to about 120 degrees, from about 70 degrees to about 110 degrees, or from about 80 degrees to about 100 degrees, from about 85 degrees to about 95 degrees, or about 90 degrees.

Embodiment 21. A method of spinal surgical planning, the method comprising:
receiving a pair of two-dimensional (2D) images of a spinal segment containing a plurality of screws, the pair of 2D images comprising a lateral view of the spinal segment and an anterior/posterior view of the spinal segment;
receiving a first set of coordinates for a first screw within the plurality of screws, the first set of coordinates including first screw feature coordinates;
generating a first 3D reconstruction of the first screw from the first set of coordinates;
receiving a second set of coordinates for a second screw within the pair of 2D images;
generating a second 3D reconstruction of the second screw from the second set of coordinates;
projecting the first 3D reconstruction and the second 3D reconstruction onto the plurality of screws within the pair of 2D images;
determining a first loss function based on alignment between the projections of the first 3D reconstruction and the second 3D reconstruction with the plurality of screws;
optimizing the first and second 3D reconstructions based on the first loss function;
identifying the orientation of the first screw and the orientation of the second screw based on the first loss function; and
predicting a shape of a rod based on the orientation of the first screw and the orientation of the second screw, the rod for surgical insertion within the spinal segment.

Embodiment 22. The method of embodiment 21, further comprising:
receiving a third set of coordinates for a third screw within the pair of 2D images;
generating a third 3D reconstruction of the third screw from the third set of coordinates;
projecting the third 3D reconstruction onto the plurality of screws within the pair of 2D images;
determining a second loss function based on alignment between the projections of the first 3D reconstruction, the second 3D reconstruction, the third 3D reconstruction, and the plurality of screws; and
identifying an orientation of the third screw based on the second loss function.

Embodiment 23. The method of embodiment 22, wherein identifying an orientation of the third screw comprises identifying an orientation of the third screw relative to the orientation of the first and second screws.

Embodiment 24. A system for predicting an orientation of a plurality of screws from a pair of two-dimensional (2D) images of a spinal segment containing the plurality of screws, the system comprising:
a display;
one or more processors in communication with the display and comprising memory capable of storing instructions executable by the one or more processors, the instructions causing the one or more processors to:
   receive a first set of coordinates for a first screw within the pair of 2D images, the first set of coordinates comprising first screw structural feature coordinates,
   receive a second set of coordinates for a second screw within the pair of 2D images, the second set of coordinates comprising second screw structural feature coordinates,
   generate a first 3D reconstruction of the first screw and the second screw from the received coordinates,
   generate a second 3D reconstruction of the first screw and the second screw from the received coordinates,
   project the first 3D reconstruction onto the pair of 2D images,
   project the second 3D reconstruction onto the pair of 2D images,
   determine a first loss function for the first 3D reconstruction based on the projection of the first 3D reconstruction onto the pair of 2D images,
   determine a second loss function for the second 3D reconstruction based on the projection of the second 3D reconstruction onto the pair of 2D images,
   adjust the first reconstruction to obtain a first minimum loss,
   adjust the second reconstruction to obtain a second minimum loss,
   identify the orientation of the first screw and the orientation of the second screw based on a comparison of the first minimum loss and the second minimum loss,
   determine rod shape instructions based on the identified orientation of the first screw and second screw, and
   send rod shape instructions to a bender, the bender for forming a rod to be placed in the spinal segment.

Embodiment 25. The system of embodiment 24, wherein the display visually overlays the first 3D reconstruction and the second 3D reconstruction on the pair of 2D images.

Embodiment 26. The system of either one of embodiments 24 or 25, wherein the instructions further cause the one or more processors to display the first 3D reconstruction over the pair of 2D images.

Embodiment 27. The system of any one of embodiments 24 through 26, wherein the instructions further cause the one or more processors to display a rod shape corresponding to the orientation of the first screw and the orientation of the second screw.

Embodiment 28. The system of any one of embodiments 24 through 27, wherein the instructions further cause the one or more processors to:
detect a change in orientation of the first screw;
generate an updated 3D reconstruction of the first screw and the second screw based on the change detected;
project the updated 3D reconstruction of the first screw and the second screw onto the pair of 2D images;
calculate an updated loss function based on projecting the updated 3D reconstruction onto the pair of 2D images; and
determine an updated rod shape based on the change in orientation of the first screw.

Embodiment 29. The system of embodiment 28, wherein the change in orientation of the first screw correlates to a changed position of a vertebrae within the spinal segment.

Embodiment 30. A method of determining an orientation of at least two screws from at least two, two-dimensional (2D) images, the method comprising:
receiving a first set of coordinates for a first screw within the 2D images, the first set of coordinates corresponding to one or more structural features of a first screw in the 2D images;
receiving a second set of coordinates for a second screw within the 2D images, the second set of coordinates corresponding to one or more structural features of the second screw in the 2D images;
generating a first 3D reconstruction of the first screw and the second screw based on the received first coordinates;
generating a first 2D projection by obtaining a profile view of the first 3D reconstruction;
generating a second 3D reconstruction of the first screw and the second screw based on the received second coordinates;
generating a second 2D projection by obtaining a profile view of the second 3D reconstruction;
displaying the first 2D projection and the second 2D projection overlaid on the at least two 2D images
receiving input from a user as to which 2D projection more closely matches the at least two 2D images;
outputting the 3D reconstruction corresponding to 2D projection selected by the user.

Embodiment 31. The method of embodiment 30, the method further comprising:
after generating the first 2D projection, comparing the first 2D projection to the 2D images to determine a first loss function and obtaining a minimum value of the first loss function by adjusting the first 3D reconstruction; and
after generating the second 2D projection, comparing the second 2D projection to the 2D images to determine a second loss function and obtaining a minimum value of the second loss function by adjusting the second 3D reconstruction.

Embodiment 32. The method of embodiment 31, wherein adjusting the first 3D reconstruction comprises iteratively adjusting the first 3D reconstruction to maximize alignment between the first 2D projection and the at least two 2D images, and wherein adjusting the second 3D reconstruction comprises iteratively adjusting the second 3D reconstruction to maximize alignment between the second 2D projection and the at least two 2D images.

Embodiment 33. The method of either one of embodiments 31 or 32, wherein adjusting the first 3D reconstruction comprises iteratively rotating the first 3D reconstruction to maximize alignment between the first 2D projection and the at least two 2D images, and wherein adjusting the second 3D reconstruction comprises iteratively rotating the second 3D reconstruction to maximize alignment between the second 2D projection and the at least two 2D images.

Embodiment 34. The method of any one of embodiments 30 through 33, wherein the method is repeated for a third screw and a fourth screw.

Embodiment 35. The method of embodiment 34, further comprising determining a rod shape based on the 3D reconstructions of the first, second, third, and fourth screws.

Embodiment 36. The method of embodiment 35, further comprising sending instructions correlated to the rod shape to a bender for forming a rod having the rod shape.

Embodiment 37. The method of any one of embodiments 30 through 36, wherein predicting the orientation of the first screw and the orientation of the second screw comprises predicting (i) a handedness, (ii) a vertical position, (iii) an angular position, and/or (iv) a rotational position for both the first screw and the second screw.

Embodiment 38. The method of any one of embodiments 30 through 37, wherein the one or more structural features comprise a screw tip, a screw shaft, a screw head, and/or a tulip.

Embodiment 39. The method of any one of embodiments 30 through 38, wherein the at least two 2D images are oriented at an angle relative to each other, the angle being from about 60 degrees to about 120 degrees, from about 70 degrees to about 110 degrees, or from about 80 degrees to about 100 degrees, from about 85 degrees to about 95 degrees, or about 90 degrees.

Embodiment 40. The method of any one of embodiments 30 through 39, wherein the at least two 2D images comprises a lateral image and an anterior/posterior image of a spinal segment of a patient.

Embodiment 41. The method of any one of embodiments 1 through 9, wherein adjusting the first 3D reconstruction comprises iteratively adjusting the first 3D reconstruction to maximize alignment between the first 2D projection and the first 3D reconstruction.

Embodiment 42. The method of embodiment 41, wherein adjusting the first 3D reconstruction comprises adjusting through differential evolution.

### Additional Terms and Definitions

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It should also be noted that some of the embodiments disclosed herein may have been disclosed in relation to a particular spinal segment (e.g., a cervical spinal segment); however, other segments (e.g., thoracic, lumbar, etc.) are also contemplated. Structures, such as a screw, can extend through the body from a surface outside the body and closer to a practitioner to a surface inside the body. Structures inside the body are referred to as more “distal” as they are further away from the practitioner, while structures that are outside of the body are referred to as “proximal.”

In one embodiment, the terms “about” and “approximately” refer to numerical parameters within 10% of the indicated range. The terms “a,” “an,” “the,” and similar referents used in the context of describing the embodiments of the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the embodiments of the present disclosure and does not pose a limitation on the scope of the present disclosure. No language in the specification should be construed as indicating any non-claimed element essential to the practice of the embodiments of the present disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Although this disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A method of determining an orientation of at least two screws from at least two, two-dimensional (2D) images, the method comprising:
receiving a first set of coordinates for a first screw within the 2D images, the first set of coordinates corresponding to one or more structural features of a first screw in the 2D images;
receiving a second set of coordinates for a second screw within the 2D images, the second set of coordinates corresponding to one or more structural features of the second screw in the 2D images;
generating a first 3D reconstruction of the first screw and the second screw based on the received first coordinates;
generating a second 3D reconstruction of the first screw and the second screw based on the received second coordinates;
comparing a first 2D projection of the first 3D reconstruction to the 2D images to determine a first loss function;
obtaining a minimum value of the first loss function by adjusting the first 3D reconstruction;
comparing a second 2D projection of the second 3D reconstruction to the 2D images to determine a second loss function;
obtaining a minimum value of the second loss function by adjusting the second 3D reconstruction;
comparing the minimum value of the first loss function to the minimum value of the second loss function to identify the loss function with a lower minimum value;
outputting the 3D reconstruction corresponding to the loss function with the lower minimum value.

2. The method of claim 1, wherein the method is repeated for a third screw and a fourth screw.

3. The method of claim 2, further comprising determining a rod shape based on the 3D reconstructions of the first, second, third, and fourth screws.

4. The method of claim 3, further comprising sending instructions correlated to the rod shape to a bender for forming a rod having the rod shape.

5. The method of claim 1, wherein predicting the orientation of the first screw and the orientation of the second screw comprises predicting (i) a handedness, (ii) a vertical position, (iii) an angular position, and/or (iv) a rotational position for both the first screw and the second screw.

6. The method of claim 1, wherein comparing the first 2D projection to the 2D images comprises visually overlaying the first 2D projection on each of the 2D images, such that the first 2D projection aligns over at least one screw within the 2D images.

7. The method of claim 1, wherein generating a first 3D reconstruction of the first screw and the second screw from the received coordinates comprises:
receiving x-ray intrinsic parameters and relative extrinsic parameters between two views;
triangulating the first screw feature coordinates;
triangulating the second screw feature coordinates; and
generating an initial 3D reconstruction of the first screw and the second screw.

8. The method of claim 1, wherein the one or more structural features comprise a screw tip, a screw shaft, a screw head, and/or a tulip.

9. The method of claim 1, wherein the at least two 2D images are oriented at an angle relative to each other, the angle being greater than about 30 degrees.

10. A method of determining an orientation of a plurality of screws from a pair of 2D images, the method comprising:
receiving a first set of coordinates for a first screw within the pair of 2D images,
the first set of coordinates derived from structural features of the first screw in the pair of 2D images, and
the pair of 2D images comprising an anterior/posterior two-dimensional image of a spinal segment and a lateral two-dimensional image of the spinal segment;
receiving a second set of coordinates derived from structural features of a second screw within the pair of 2D images;
generating a first three-dimensional (3D) reconstruction of the first screw and the second screw from the received first coordinates;
generating a second 3D reconstruction of the first screw and the second screw from the received second coordinates;
projecting the first 3D reconstruction of the first screw and the second screw onto the pair of 2D images, such that the first 3D reconstruction is projected over the plurality of screws within the pair of 2D images;
projecting the second 3D reconstruction of the first screw and the second screw onto the pair of 2D images, such that the second 3D reconstruction is projected over the plurality of screws within the pair of 2D images;
calculating a first loss function for the projection of the first 3D reconstruction based on alignment of the projection and the plurality of screws within the pair of 2D images;
obtaining a minimum value of the first loss function by adjusting the first 3D reconstruction;
calculating a second loss function for the projection of the second 3D reconstruction based on alignment of the projection and the plurality of screws within the pair of 2D images;
obtaining a minimum value of the second loss function by adjusting the second 3D reconstruction;
comparing the minimum value of the first loss function and the minimum value of the second loss function and selecting the 3D reconstruction with the lower loss function; and
identifying, based on the 3D reconstruction with the lower loss function, an orientation of the first screw and an orientation of the second screw within the pair of 2D images.

11. The method of claim 10, wherein generating a first three-dimensional (3D) reconstruction of the first screw and the second screw from the received first coordinates and the received second coordinates comprises generating a gradient of 2D images of the first screw and the second screw.

12. The method of claim 11, wherein calculating a first loss function comprises:
measuring a similarity of the gradient of 2D images of the first screw and the second screw with a gradient of the pair of 2D images; and
computing a similarity score between both gradients for consistency.

13. The method of claim 10, wherein identifying the orientation of the first screw and the orientation of the second screw within the pair of 2D images comprises visually matching a 3D reconstruction of the first screw with the first screw within the pair of 2D images and visually confirming a 3D reconstruction of the second screw with the second screw within the pair of 2D images.

14. The method of claim 10, wherein the method does not require manual identification of the first set of coordinates or the second set of coordinates.

15. The method of claim 10, wherein calculating a first loss function comprises generating an image gradient of a projection of the first 3D reconstruction and an image gradient of the pair of 2D images.

16. The method of claim 15, wherein calculating a first loss function comprises:
measuring a similarity between the image gradient of the projection of the first 3D reconstruction and the image gradient of the pair of 2D images; and
computing a similarity score between both gradients for consistency.

17. A method of spinal surgical planning, the method comprising:
receiving a pair of two-dimensional (2D) images of a spinal segment containing a plurality of screws, the pair of 2D images comprising a lateral view of the spinal segment and an anterior/posterior view of the spinal segment;
receiving a first set of coordinates for a first screw within the plurality of screws, the first set of coordinates including first screw feature coordinates;
generating a first 3D reconstruction of the first screw from the first set of coordinates;
receiving a second set of coordinates for a second screw within the pair of 2D images;
generating a second 3D reconstruction of the second screw from the second set of coordinates;
projecting the first 3D reconstruction and the second 3D reconstruction onto the plurality of screws within the pair of 2D images;
determining a first loss function based on alignment between projections of the first and second 3D reconstructions and the plurality of screws in the pair of 2D images;
optimizing the first and second 3D reconstructions based on the first loss function;
identifying the orientation of the first screw and the orientation of the second screw based on the first loss function; and
predicting a shape of a rod based on the orientation of the first screw and the orientation of the second screw, the rod for surgical insertion within the spinal segment.

18. The method of claim 15, further comprising:
receiving a third set of coordinates for a third screw within the pair of 2D images;
generating a third 3D reconstruction of the third screw from the third set of coordinates;
projecting the third 3D reconstruction onto the plurality of screws within the pair of 2D images;
determining a second loss function based on alignment between the projections of the first 3D reconstruction, the second 3D reconstruction, the third 3D reconstruction, and the plurality of screws; and
identifying an orientation of the third screw based on the second loss function.

19. The method of claim 16, wherein identifying an orientation of the third screw comprises identifying an orientation of the third screw relative to the orientation of the first and second screws.
